(19) United States
(12) Patent Application Publication
(10) Pub. No.: US 2004/0214839 A1
(43) Pub. Date: Oct. 28, 2004

(54) Therapeutic methods

(76) Inventor: Rodney William Kelly, Edinburgh (GB)

Correspondence Address: Michael L Goldman, Nixon Peabody, Clinton Square, PO Box 31051, Rochester, NY, 14603, US

(21) Appl. No.: 10/477,170

(22) PCT Filed: May 10, 2002
(86) PCT No.: PCT/GB02/02114

(30) Foreign Application Priority Data
May 11, 2001 (GB) 0111497.4

(51) Int. Cl. A61K 31/522; A61K 31/4015; A61K 31/557

(52) U.S. Cl. 514/263.36; 514/424; 514/573

(57) ABSTRACT

A method of inducing tolerance to an antigen in a patient, the method comprising administering to the patient a prostaglandin or agonist thereof and a type IV selective phosphodiesterase (PDE) inhibitor.

### THERAPEUTIC METHODS

The present invention relates to therapeutic methods and uses; in particular it relates to methods for inducing tolerance to an antigen in a patient.

An organism's immunity to an antigen arises as a consequence of a first encounter with the antigen and the subsequent production of immunoglobulin molecules, for example, antibodies, capable of selectively binding that antigen. In addition, the immune response is controlled by T cells which may be antigen specific. Immunity allows the rapid recruitment, usually by stimulating an inflammatory response, of cells which can dispose of the foreign antigen. Under certain circumstances, the immune system does not produce an immune response against antigens due to a mechanism called “tolerance”. For example, an immune system can normally discriminate against foreign antigens and constituents of the organism itself, due to a mechanism whereby all B lymphocytes which could potentially produce antibodies to constituents of the organism itself (“self antigens”) are destroyed during development, thereby removing the organism's capacity to produce antibodies directed to a self antigen.

Tolerance is probably an active process. This means that peripheral tolerance is gained where an antigen is presented to a T cell in a particular environment (eg high IL-10 levels and low IL-12 levels). The T cells then circulate and when they meet that specific antigen again they do not mount an immune response (anergic T cells) or they mount a quelling response (regulatory T cells). A role for regulatory T cells has been proposed in tolerance. The regulatory T cells are programmed by the environment of the antigen presenting cell to react to their cognate antigen by releasing “down-regulatory” cytolines. The first such regulatory cells described were induced by IL-10 (Groux et al., 1997, Nature 389:737-742).

Where tolerance breaks down, the organism may produce a cellular immune response (including cytotoxic T cells) to normal constituents of the organism, producing an “autoimmune disease”. Examples of autoimmune diseases include systemic lupus erytiematosus (SLE), multiple sclerosis (MS) and Hashimoto's disease.

In some circumstances, even the normal response of the immune system to a foreign antigen can produce undesirable results, such as in the case of tissue or organ grafts or transplants, where the immune system of the tissue or organ recipient recognises the tissue or organ graft or transplant as foreign and acts to reject it.

One of the drawbacks of existing methods of treating immune or inflammatory conditions or diseases however, is the limited range of options and their therapeutic inadequacy. For example, glucocorticosteroids used for treating inflammatory respiratory disease have toxic effects in many patients, and alternatives such as cyclosporin A or interferon τ are high-risk, expensive and generally unsatisfactory.

Unexpectedly, the inventor has found that there is a synergistic effect between a prostaglandin and a type IV selective phosphodiesterase (PDE) inhibitor on the release of interleukin-10 (IL-10) from cells of the immune system. Furthermore, the inventor has found that there is a marked stimulation of IL-10 and inhibition of interleukin-12 (IL-12) in cells of the immune system when a prostaglandin and a type IV selective PDE inhibitor are used in combination. In the presence of a type IV selective PDE inhibitor, the stimulation of IL-10 by both PGE and 19-hydroxy PGE was increased strikingly.

Type IV selective PDE inhibitors such as Rolipram are known to raise cAMP and IL-10 levels in monocyte/macrophages stimulated with the bacterial coat product lipopolysaccharide (LPS) (Strassman et al., 1994 J. Exp. Med. 180: 2365-70; Kraan et al., 1995 J. Exp. Med. 181: 775-9; Kambayashi et al., 1995 J. Immunol. 155: 4909-16). Unexpectedly, the inventor has found that there is a synergistic effect between prostaglandin and a type IV selective PDE inhibitor on the release of IL-10 from cells of the immune system, which results in a dramatic increase in the release of IL-10.

The inventor also shows an increase in PDE activity that follows both PGE and 19-hydroxy PGE application. This is a direct negative feedback to reduce the effect of the stimulus. Use of a PGE and a type IV selective PDE inhibitor increases PDE message even further, but then the synthesised phosphodiesterase is nullified by the presence of the inhibitor.

In diseases resulting from an aberrant or undesired immune response there is often a deficiency in IL-10 and/or an increase in IL-12. This imbalance in IL-10 may be detrimental to the development of useful T helper cells, particularly T regulatory cells; a preponderance of type 1T helper cells over type 2T helper cells is thought to be characteristic of autoimmune disease. Thus, stimulation of IL-10 production and inhibition of IL-12 is believed to induce a tolerising environment for T cell activation.

The inventor now proposes the use of a type IV selective PDE inhibitor in combination with a prostaglandin or agonist thereof in the induction of tolerance of, or tolerance to, an antigen in a patient.

Furthermore, the combination of a type IV selective PDE inhibitor and a prostaglandin or agonist thereof is considered by the inventor to achieve the desirable effect of reducing the amount of prostaglandin or agonist thereof or PDE inhibitor required to achieve a useful degree of therapeutic benefit, and/or reducing the side effects of administration of prostaglandin or agonist thereof.

As far as the inventor is aware, there has never been any suggestion that a combination of a prostaglandin or agonist thereof and a type IV selective inhibitor of PDE could be used to stimulate IL-10 production, and there has been no suggestion of a treatment using this combination to stimulate IL-10. Furthermore, there has never been any suggestion that this combination could be used to inhibit IL-12 production, or to induce a tolerising environment for T cell activation, or to induce tolerance to an antigen in a patient.

The use of a combination of a prostaglandin and a PDE inhibitor to alleviate the symptoms of psoriasis and related proliferative slin disorders has been suggested in U.S. Pat. No. 4,034,087, without actually providing any examples of a prostaglandin and a PDE inhibitor being used to treat them. Such an application of a PGE and PDE inhibitor is anti-inflammatory rather than immunomodulatory.

The principal receptor for prostaglandin E2 (PGE2) are the EP₂ and EP₄ sub-types, however other receptor sub-types exist, namely EP₁ and EP₃. EP₂ and EP₄ receptors couple with adenylcyclase and use elevated cAMP as the messenger system. The levels of cAMP in tissue are governed both by synthesis and by catabolism by PDE. PDE can be blocked by specific inhibitors. The inventor believes, but without being bound by any theory, that the administration of a type IV selective PDE inhibitor will enhance the effect of a prostaglandin or agonist thereof in inducing tolerance to an antigen in a patient. Thus, the inventor believes, but without being bound by any theory that the effect of a prostaglandin or agonist thereof (such as PGE) acting on its EP₂ and EP₄ receptors is to stimulate cAMP and the addition of the type IV selective PDE inhibitor provides a synergistic action on monocytes and macrophages resulting in a reduction in the immune response which is greater than the effect of the sum of the same amount of either prostaglandin or agonist thereof or type IV selective PDE inhibitor administered alone.

A first aspect of the invention provides a method of inducing tolerance to an antigen in a patient, the method comprising administering to the patient a prostaglandin or agonist thereof and a type IV selective PDE inhibitor.

By inducing tolerance to an antigen we include the meaning that the immune system of the patient may become tolerant of an antigen where it was intolerant before, or the immune system may mount a reduced response or no response at all (ie, an undetectable response) to an immune stimulus such as an antigen.

An effect of the treatment of a patient with a prostaglandin or agonist thereof and a type IV selective PDE inhibitor may be the facilitation or improvement of tolerance to an antigen. The antigen may be one which is foreign to the patient, such as an antigen which is involved in irritable bowel syndrome.

It will also be appreciated that the induction of tolerance to an antigen in a patient upon administration of prostaglandin or agonist thereof and a type IV selective PDE inhibitor may lead to antigen specific immune suppression. Thus, the invention includes a method of inducing tolerance to an antigen in a patient to create a state of immune suppression in the patient, the method comprising administering to the patient a prostaglandin or agonist thereof and a type IV selective PDE inhibitor. Such a state of immune suppression is characterised by raising the threshold of a cell-mediated immune response to any antigenic stimulus.

Thus, it will be seen that the invention also provides the use of the combination of a prostaglandin or agonist thereof and a PDE inhibitor as an immunosuppressant.

The invention therefore includes suppressing the immune system in a patient. By “suppressing” we include the meaning that the immune system response is altered such the immune system mounts a reduced response or no response to an immune stimulus. Accordingly, the invention includes inducing tolerance to an antigen in a patient leading to amelioration of an aberrant or undesired immune response in the patient.

The method of the invention also includes inducing tolerance to an antigen in a patient for the treatment of diseases or conditions where there is an undesirable immune response.

The invention includes a method of treating an autoimmune disease in a patient, the method comprising administering to the patient a prostaglandin or agonist thereof and a type IV selective PDE inhibitor. Such autoimmune diseases include primary myxoedema, thyrotoxicosis, pernicious anaemia, autoimmune atrophic gastris, Addison's disease, insulin-dependent diabetes mellitus (IDDM), Goodpasture's syndrome, myasthenia gravis, sympathetic ophthalmia, MS, autoimmune haemolytic anaemia, idiopathic leucopenia, ulcerative colitis, dermatomyositis, scleroderma, mixed connective tissue disease, rheumatoid arthritis, irritable bowel syndrome, SLE, Hashimoto's disease, thyroiditis, Behcet's disease, coeliac disease/dermatitis herpetiformis, and demyelinating disease.

In an alternative embodiment, the invention includes a method of treating an autoimmune disease, with the exception of rheumatoid arthritis, in a patient, the method comprising administering to the patient a prostaglandin or agonist thereof and a type IV selective PDE inhibitor.

The treatment is believed to combat the undesirable autoimmune response directly, as well as treating the symptoms by directing T cells away from a pro-inflammatory role.

Without being bound by theory, the inventor believes that the methods of the present invention may affect the programming of T cells so that they become regulatory or suppressive T cells rather than pro-inflammatory T cells. When a T cell meets an antigen, in the presence of a prostaglandin and type IV selective PDE inhibitor, it will release a suppressive cytokine such as IL-10 and not an inflammatory cytokine such as IL-12. Treatment with a prostaglandin and type IV selective PDE inhibitor is thus believed to prevent or minimise an inflammatory response from developing. Thus treatment with a prostaglandin and type IV selective PDE inhibitor can be used prophylactically, or as soon as the first symptoms of, eg an autoimmune disease, appear. Furthermore, it will be appreciated that because T cells are present throughout the body they may be programmed or primed at a site remote from their ultimate site of action. Similarly, unlike other forms of treatment of certain autoininune diseases, the method may be helpful in preventing inflammatory responses before they start. Thus, the method may be useful in treating patients who, for example because of their age or genetic factors, are predisposed to an autoimmune disease before any inflammatory symptoms show.

Thus, the invention also includes inducing tolerance to an antigen in a patient for inhibiting or dampening an immune or inflammatory response in the patient. By “inhibition or dampening” we include increasing the level of IL-10, and/or decreasing the level of IL-12 which leads to an increase in the Th2 response a decrease in the Th1 response

The invention also includes inducing tolerance to an antigen in a patient leading to amelioration of an aberrant or undesired inflammatory response in the patient. The aberrant or undesired inflammator response is not associated with psoriasis, or a related proliferative skin disorder.

By “aberrant or undesired immune or inflammatory response” we include diseases or conditions which cause the presence of visible or measurable inflammation within a tissue in an individual or patient. For example, the tissue that forms part of an allograft or the tissues of a host having received an allograft, or the central nervous system of an individual with MS, or insulitis in a patient with type 1 diabetes, swollen joints in a patient with rheumatoid arthritis.

The invention includes a method of inducing tolerance to an antigen in a patient thereby suppressing an aberrant or undesired immune or inflammatory response in the patient, such as a response related to transplant rejection.

The invention therefore includes the treatment of a disease or condition associated with transplant rejection such as graft versus host disease or host versus graft disease, for example in organ or skin transplants. In these cases, an inhibition or dampening of an immune or inflammatory response may be required. Thus, the invention includes the combating of transplant rejection.

Diseases or conditions where there is an aberrant or undesired immune or inflammatory response may also include allergies, wherein the undesired response is an allergic response. In such a condition or disease, the antigen to which tolerance is induced would be an allergen.

Thus, the invention includes a method of treating an allergic condition or disease in a patient, the method comprising administering to the patient a prostaglandin or agonist thereof and a type IV selective PDE inhibitor.

The allergy may be any allergy such as allergy to cat dander, house dust mite, grass or tree pollens, fungi, moulds, foods, stinging insects and so on.

In one preferred embodiment, the allergic condition or disease is allergic asthma, and the PG and the type IV selective PDE inhibitor are preferably administered to the lungs or bronchial tree, preferably via an aerosol. This embodiment may be particularly advantageous as some 19-hydroxy prostaglandin analogues have been reported to function as bronchodilators, such as those described in U.S. Pat. No. 4,127,612, incorporated herein by reference. The reason why PGs are not widely used in the treatment of asthma is that they make the patient cough. Administration of a type IV selective PDE inhibitor would allow the PG to be administered at a lower concentration, thus providing the therapeutic benefits while minimising the side-effects.

Thus the invention includes the use of a PGE or 19-hydroxy PGE and a type IV selective PDE inhibitor for treatment by inhalation of asthma due to allergy.

Whether or not a particular patient is one who is expected to benefit from treatment may be determined by the physician.

The prostaglandin or agonist thereof and the type IV selective PDE inhibitor may be administered in any order. Preferably, they are co-administered. However, they may be administered so that the type IV selective PDE inhibitor can take effect in the accessory cells prior to administration of the prostaglandin or agonist thereof. The prostaglandin or agonist thereof and the type IV selective PDE inhibitor may be administered substantially simultaneously, for example in the same composition. The order and timing of administration may be determined by the physician using knowledge of the properties of the prostaglandin and type IV selective PDE inhibitor. For example, the prostaglandin (such as misoprostol) may be active over a period of 4 hours following administration. The type IV selective PDE inhibitor may take of the order of 30 minutes to take effect after administration. Thus, suitable timings of administration can readily be devised from this information.

Where the tolerance to an antigen is desired to be localised to a particular organ, for example to the skin or the bronchial tree and lungs, it is preferred if the prostaglandin or agonist thereof is administered locally at the site of the condition. The prostaglandin or agonist thereof may be administered as a gel or cream or vapour or spray or in a “patch” in the case of a condition localised to the skin, or as an inhaled vapour or spray where the site is the lungs or bronchial tree.

As is described in more detail below, the prostaglandin or agonist thereof may be administered systemically, such as orally. For example, the prostaglandin or agonist thereof may be administered to the mucosal immune system, eg via a suppository, and is expected to act at mucosal sites remote from administration.

The type IV selective PDE inhibitor may be administered by any suitable route. The type IV selective PDE inhibitor may reach the desired site of inhibition of type IV PDE, which is typically the leukocytes in relation to the present invention using many different routes of administration. Typically, in one embodiment, the type IV selective PDE inhibitor is administered systemically. Suitable forms of systemic administration include oral, transcutaneous or by suppository. The type IV selective PDE inhibitor may be administered to the mucosal immune system, eg via a suppository, and is expected to act at mucosal sites remote from administration. Type IV selective PDE inhibitors are orally available, so it may be convenient to administer the type IV selective PDE inhibitor orally.

It is also convenient to administer the type IV selective PDE inhibitor locally. Thus, the type IV selective PDE inhibitor may be delivered locally, such as on the skin, using, for example, a gel or cream or vapour or spray or in a “patch” as described above in relation to the administration of the prostaglandin or agonist thereof. Similarly, in the case of administration to the bronchial tree or lungs it may be administered as a spray or vapour.

In preferred embodiments of the invention, the prostaglandin or agonist thereof and the type IV selective PDE inhibitor may be combined in the same formulation for delivery simultaneously. Thus, the prostaglandin or agonist thereof and the PDE inhibitor may be combined in a gel or a cream or a vapour or spray or “patch” or suppository and administered together to the patient.

In a preferred embodiment, a suppository containing PGE or 19 hydroxy PGE and a type IV selective PDE inhibitor has an enteric coating which only releases the active agents in the bowel when the pH has risen. This sort of preparation has been successful in the delivery of glucocorticoids to the bowel (data sheet for Entocort CR).

Alternatively, the PG and PDE inhibitor can be administered in a capsule or other suitable form that is swallowed. The capsule or other suitable has an enteric coating which is pH sensitive leading to release at an appropriate point in the gastrointestinal tract where it is desired to do so, typically the distal ileum or colon.

Alternatively, the prostaglandin and/or type IV selective PDE inhibitor may be administered directly to the colon or distal ileum via a non-soluble tube or pipe system, such as produced by Egalet.

A suppository containing PGE or 19 hydroxy PGE and a type IV selective PDE inhibitor may be effective for treating inflammatory bowel disease, which can be caused by antigen-specific immune responses (Groux et al).

The administration of prostaglandin and type IV selective PDE inhibitor to a mucosal site remote from the site of inflammation, eg co-administration as a suppository in the treatment of arthritis, may be particularly advantageous as pathologic changes in the gastrointestinal tract can be associated with clinical complaints in multiple organs, including the musculoskeletal system (Alghafeer & Sigal, Bulletin on the Rheumatic Diseases, 51(2): http://www.arthritis.org/research/bulletin/vol51no2/51_{—}2_printable.asp, incorporated herein by reference). Some reactive arthritis can be triggered by inflammatory bowel diseases, and lymphocytes from the gut mucosa have been reported to migrate to joint tissue in enteropathic arthritis (Salmi & Jalkanen (2001) J. Immunol., 166(7):4650-7, incorporated herein by reference).

The prostaglandin or agonist thereof may be any suitable prostaglandin or agonist thereof. By “prostaglandin or agonist” we mean any compound which acts as a prostaglandin agonist on a prostaglandin receptor. The prostaglandin agonist need not be a prostanoid. Typically, the agonist is one which binds the EP₂ or EP₄ receptor. It is preferred that the prostaglandin or agonist thereof is one which is able to stimulate cAMP production in macrophages. It is preferred that the prostaglandin is a PGE or a PGI. Preferably, the prostaglandin is not a PGF or agonist thereof. It is preferred that the prostaglandin or agonist thereof is PGE₂ or a synthetic analogue thereof. Synthetic analogues include those modified at position 15 or 16 by the addition of a methyl group or those where the hydroxyl has been transposed from position 15 to position 16. Preferred examples of analogues of prostaglandin include Butaprost (an EP₂ receptor agonist) and 11-deoxy PGE1 (an EP₄ receptor agonist). For the avoidance of doubt, the term “prostaglandin” includes naturally-occurring prostaglandins as well as synthetic prostaglandin analogues.

Suitable prostaglandins or agonists thereof include dinoprostone (sold as Propess by Ferring in Europe and Forest in the USA; sold as Prostin E2 by Pharmacia), gemeprost (sold by Farillon), misoprostol (which is sold as Cytotec by Searle and Pharmacia), alprostadil (which is sold as Caverject by Pharmacia and Viridal by Schwarz and MUSE by AstraZeneca) and limaprost.

Misoprostol is a PGE analogue which has EP2 and EP3 agonist effects. Its chemical structure is (±) methyl 11α, 16-dihydroxy-16-methyl-9-oxoprost-13-enoate.

An example of a non-prostanoid compound which acts as a prostaglandin agonist is AH23848, an EP4 receptor agonist.

EP2 agonists which may be useful in the practise of the invention include AH13205.

Suitable prostaglandins thereof also include 19-hydroxy PGE1 and 19-hydroxy PGE2. Prostaglandin agonists are described in EP 1 097 922 and EP 1 114 816, incorporated herein by reference.

Suitable prostaglandins or agonists thereof may also include any of the 19-hydroxy prostaglandin analogues described in U.S. Pat. No. 4,127,612, incorporated herein by reference.

It is preferred that the prostaglandin is prostaglandin E₂ (PGE₂). Prostaglandins and agonists thereof, including PGE₂, are commercially available, for example from Pharmacia and Upjohn as Prostin E2.

The type IV selective PDE inhibitor may be any suitable type IV selective PDE inhibitor. Preferably, the type IV selective PDE inhibitor inhibits type IV PDEs which are known to be active in cAMP breakdown. By “selective” we mean that the inhibitor inhibits type IV PDE more potently than another type. Preferably, the type IV selective inhibitor is at least 2 times more potent an inhibitor of type IV PDE than another PDE type. More preferably, the type IV selective inhibitor is at least 5 times, 10 times, 20 times, 30 times, 40 times, 50 times, 100 times, 200 times, 500 times or 1000 times more potent an inhibitor of type IV PDE than another PDE type. Typically, the inhibitor is around 5 to 50 times more potent an inhibitor of the PDE type IV than another PDE type. Typically, the inhibitor is 5 to 50 times more potent an inhibitor of type IV PDE than an inhibitor that is considered to be non-selective such as theophylline. Thus, theophylline is 30 times less effective than rolipram.

Preferably, selective inhibition is determined by a comparison of IC₅₀ levels (Dousa (1999) Kidney International 55: 29-62).

U.S. Pat. No. 6,127,378 discloses phenanthridines substituted in the 6 position that are described as selective PDE inhibitors (mainly of type IV), that may be suitable for use in the methods of the invention. The disclosure of U.S. Pat. No. 6,127,378 relating to type IV selective PDE inhibitors is incorporated herein by reference.

Specific (or selective) type IV PDE inhibitors include rolipram (4-[3-cyclopentyloxy 4-methoxyphenyl]-2-pyrrolidinone) and Ro-20-1724 (4-[3-butoxy-4-methoxybenzyl]-2-imidazolidinone). The IC₅₀ for rolipram is 800 nM, and the IC₅₀ for Ro-20-1724 is 2 μM.

Another suitable PDE type IV selective inhibitor is denbufylline (1,3-di-n-butyl-7-(2-oxopropyl)-xanthine).

CP 80 633 (Hanifin et al (1996) J. Invest. Dermatol. 107, 51-56), CP 102 995 and CP 76 593 are also all potent type IV inhibitors (available from Central Research Division, Pfizer Inc, Groton, Conn.).

Other high affinity type IV selective PDE inhibitors include CPD 840, RP 73401, and RS 33793(Dousa, 1999). The high affinity type IV selective PDE inhibitors have a Kᵢ of approximately 1 nM while the lower affinity inhibitors have a Kᵢ of about 1 μM.

The disclosures in Dousa (1999); Müller et al (1996, Trends Pharmacol. Sci. 17: 294-298); Palfreyman & Souness (1996, Prog Med Chem 33:1-52); Stafford & Feldman (1996, Annual Reports in Medicinal Chemistry (vol 31) pp 71-80; Ed. Bristol, Academic Press, NY, USA); and Teixeira et al (1997, Trends Pharmacol. Sci. 18: 164-171) relating to type IV PDE selective inhibitors are incorporated herein by reference.

Typically, when a type IV PDE-selective inhibitor is administered orally, around 1 to 30 mg is used. Thus, a typical oral dose of rolipram or denbufylline is 1 mg or 5 mg or 10 mg or 30 mg.

In one embodiment the prostaglandin or agonist thereof is administered orally. In particular the prostaglandin or agonist thereof is a prostaglandin analogue which has been modified to reduce its catabolism and which is orally available (such as misoprostol).

Although the type IV selective PDE inhibitor can be administered by any suitable means and by any suitable route, when the prostaglandin or agonist thereof is administered orally it is preferred that the type IV selective PDE inhibitor is also administered orally. It is also preferred if the prostaglandin or agonist thereof and type IV selective PDE inhibitor are administered simultaneously, for example in the same composition.

Thus, in a preferred embodiment, the method of the invention makes use of the oral administration of a prostaglandin analogue which has been modified to reduce its catabolism and which is orally available (such as misoprostol) and the oral administration of the type IV selective PDE inhibitor, such as rolipram. The advantages of oral administration is that it generally has good compliance compared to other modes of administration.

The inventor believes that the combination of type IV selective PDE inhibitor with the orally available prostaglandin or agonist thereof will mean that a lower dose of oral prostaglandin will be required than in the absence of the type IV selective PDE inhibitor. It is believed by the inventor that this will have the advantage of reducing side effects caused by the oral prostaglandin or agonist thereof, such as muscle cramps.

Typically, 0.1-100 μg of 19 hydroxy PGE and 1-250 μg Rolipram in 5 ml saline would be administered.

Typically, 100 to 800 μg of misoprostol is administered orally daily with 1 to 30 mg of rolipram or denbufylline.

As described above, the prostaglandin or agonist thereof can be used orally in combination with a PDE inhibitor at a lower dose than in the absence of PDE inhibitor.

Typically, the dose of type IV selective PDE inhibitor is as described above and the prostaglandin, such as misoprostol, is administered at a dose of 100 to 400 μg.

The data described in the Figures and Examples shows that typically a higher concentration of 19-hydroxy PGE would be necessary to achieve similar effects to PGE. However, 19 hydroxy PGE has the advantage of being more rapidly catabolised.

Thus, preferably, the combination of a type IV selective PDE inhibitor and prostaglandin or agonist thereof, comprises a selective type IV PDE inhibitor and a 19-hydroxy PGE.

A second aspect of the invention provides the use of a prostaglandin or agonist thereof in the manufacture of a medicament for inducing tolerance to an antigen in a patient wherein the patient is administered a type IV selective PDE inhibitor. Thus, the patient may already have been administered the type IV selective PDE inhibitor before administration of the prostaglandin or agonist thereof, or is administered the type IV selective PDE inhibitor at the same time as the prostaglandin or agonist thereof or will be administered the type IV selective PDE inhibitor after administration of the prostaglandin or agonist thereof.

A third aspect of the invention is the use of a type IV selective PDE inhibitor in the manufacture of a medicament for inducing tolerance to an antigen in a patient wherein the patient is administered a prostaglandin or agonist thereof. Thus, the patient may already have been administered the prostaglandin or agonist thereof before administration of the type IV selective PDE inhibitor, or is administered the prostaglandin or agonist thereof at the same time as the type IV selective PDE inhibitor or will be administered the prostaglandin or agonist thereof after administration of the type IV selective PDE inhibitor.

A fourth aspect of the invention provides the use of a combination of a prostaglandin or agonist thereof and a type IV selective PDE inhibitor in the manufacture of a medicament for inducing tolerance to an antigen in a patient. Thus, the prostaglandin or agonist thereof and type IV selective PDE inhibitor may be combined in the same medicament before administration to the patient.

Preferably, the use according to the second, third and fourth aspects is in treating an aberrant or undesired immune or inflammatory response in the patient.

The preferences for the prostaglandin or agonist thereof, type IV selective PDE inhibitors, routes of administration, doses and so on for the second, third and fourth aspects of the invention are the same as for the first aspect of the invention.

A fifth aspect of the invention provides a therapeutic system for inducing tolerance to an antigen, the system comprising a prostaglandin or agonist thereof and a type IV selective PDE inhibitor. The therapeutic system may also be termed a “kit of parts”.

Preferably, the therapeutic system contains a preferred prostaglandin or agonist thereof as defined in the first aspect of the invention. Still preferably, the therapeutic system contains a preferred type IV selective PDE inhibitor as defined in the first aspect of the invention. The therapeutic system or kit of parts may suitably contain both the prostaglandin or agonist thereof and the type IV selective PDE inhibitor packaged and presented in suitable formulations for use in combination, either for administration simultaneously or for administration which is separated in time. Thus, for example, in one embodiment where the prostaglandin or agonist thereof and type IV selective PDE inhibitor are for simultaneous administration locally to the skin, the therapeutic system may contain a gel or cream or spray or vapour or “patch” which contains a combination of prostaglandin or agonist thereof and PDE inhibitor. Alternatively, in another embodiment where the prostaglandin or agonist thereof and type IV selective PDE inhibitor are for separate administration in a particular treatment regime, the prostaglandin or agonist thereof and PDE inhibitor are packaged or formulated separately. For example, the prostaglandin or agonist thereof may be formulated for administration locally using a cream or gel or spray or vapour or “patch”, and the type IV selective PDE inhibitor is packaged or formulated for systemic administration such as oral administration.

The formulations of the prostaglandin or agonist thereof alone or type IV selective PDE inhibitor alone or the combination thereof may conveniently be presented in unit dosage form and may be prepared by any of the methods well known in the art of pharmacy. Such methods include the step of bringing into association the active ingredients used in the invention with the carrier which constitutes one or more accessory ingredients. In general the formulations are prepared by uniformly and intirnately bringing into association the active ingredient with liquid carriers or finely divided solid carriers or both, and then, if necessary, shaping the product.

Formulations in accordance with the present invention suitable for oral administration (eg of the type IV selective PDE inhibitor or of a suitable prostaglandin or agonist thereof) may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

For local administration to the skin, it may be convenient to formulate the prostaglandin or agonist thereof and/or type IV selective PDE inhibitor in combination with a dispersion agent or an agent which allows for increased transdermal or transmucosal transfer or penetration, such a dimethyl sulphoxide (DMSO) and the like. Suitable agents are ones which are compatible with the prostaglandin or agonist thereof and/or type IV selective PDE inhibitor (eg are solvents thereof).

A composition comprising a prostaglandin or agonist thereof and a type IV selective PDE inhibitor is useful in the practice of the invention.

Typically, the composition is packaged and presented for use in medicine. The composition may be used in human or veterinary medicine; preferably, it is used in human medicine.

Typically, the composition further comprises a pharmaceutically acceptable carrier. Thus, a pharmaceutical composition (or formulation as it may be termed) comprising a prostaglandin or agonist thereof, a type IV selective PDE inhibitor and a pharmaceutically acceptable carrier is useful in the practice of the invention. The carrier(s) must be “acceptable” in the sense of being compatible with the composition of the invention and not deleterious to the recipients thereof. Typically, the carriers will be water or saline which will be sterile and pyrogen free.

The patient on which the method or medicament is used is preferably a human although the patient may be any mammal such as a cat, dog, horse, cow, sheep, horse, pig and so on.

It will be appreciated that the method or medicament may be used before symptoms indicating a need to induce tolerance of an antigen becomes apparent in the patient to be treated, or, either alternatively or in addition, the using of the method or medicament may be used after symptoms or signs become apparent. Thus, in the case of a patient receiving an organ or tissue transplant, it may be beneficial to administer the prostaglandin or agonist thereof and type IV selective PDE inhibitor before the transplantation surgery is started. It may be furler beneficial to continue the administration during or after completion of the transplant or graft surgery. The necessary dosage may be determined by the physician, according to the degree of tolerance that is required.

It will flirier be appreciated that each of the prostaglandin or agonist thereof, the type IV selective PDE inhibitor may be administered as a single dose, or in multiple smaller doses which achieve the same therapeutic effect. The frequency of administration may vary according to the convenience of the physician administering the dose or the patient.

Pregnancy is likely to be a contraindication for the present invention. In fact, pregnancy is a contraindication for several prostaglandins including misoprostol. Cytotec (misoprostol) does not cause hypotension, but this may be a possible risk with the method of the invention.

The invention will now be described in more detail with the aid of the following Figures and Examples.

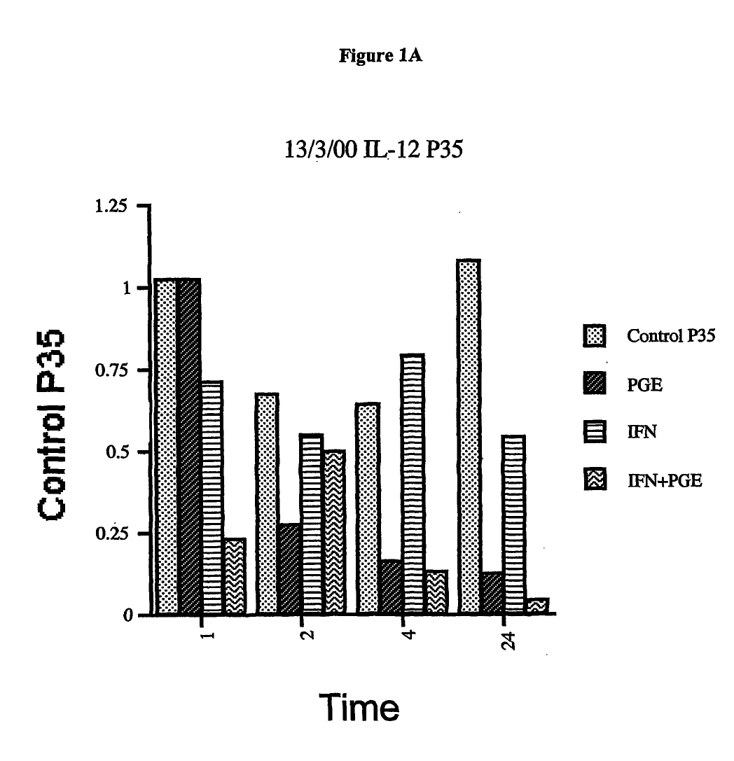

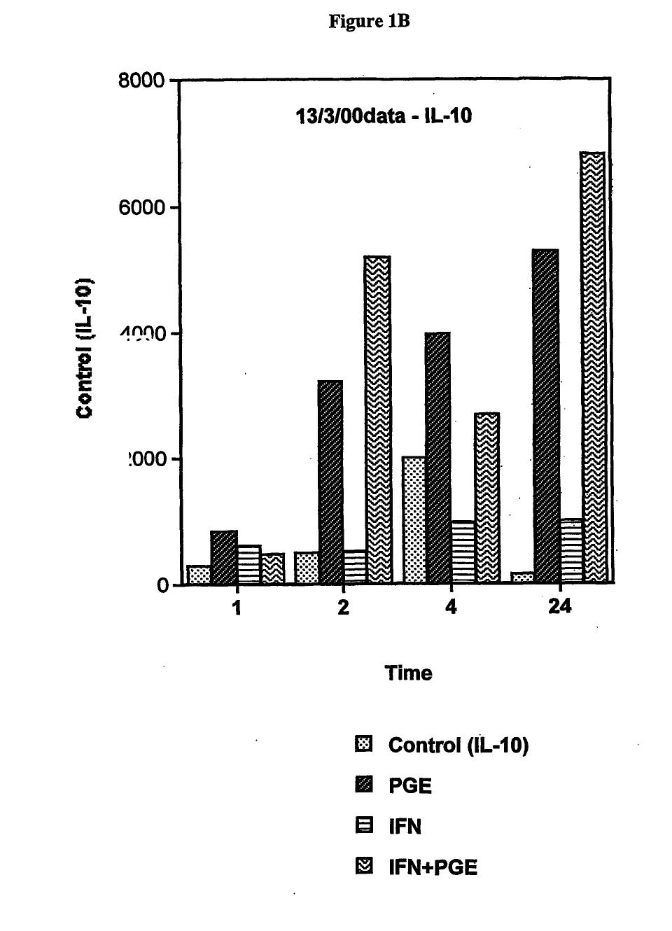

FIG. 1

Expression of mRNA for cytokines IL-10 and IL-12 subunit p35. Experiment carried out on U937 cells (pro-monocytes) in the presence of Rolipram at 1 μg/ml=4 μM and indomethacin 10 μM. The indomethacin prevents prostaglandin synthesis from cells. Note that the effect of PGE+Rolipram is a marked stimulation of IL-10 and an inhibition of IL-12 both for unstimulated and IFNγ stimulated cells. Vertical scale is a measure of mRNA compared to a control sample as measured by real-time quantitative PCR (Taqman).

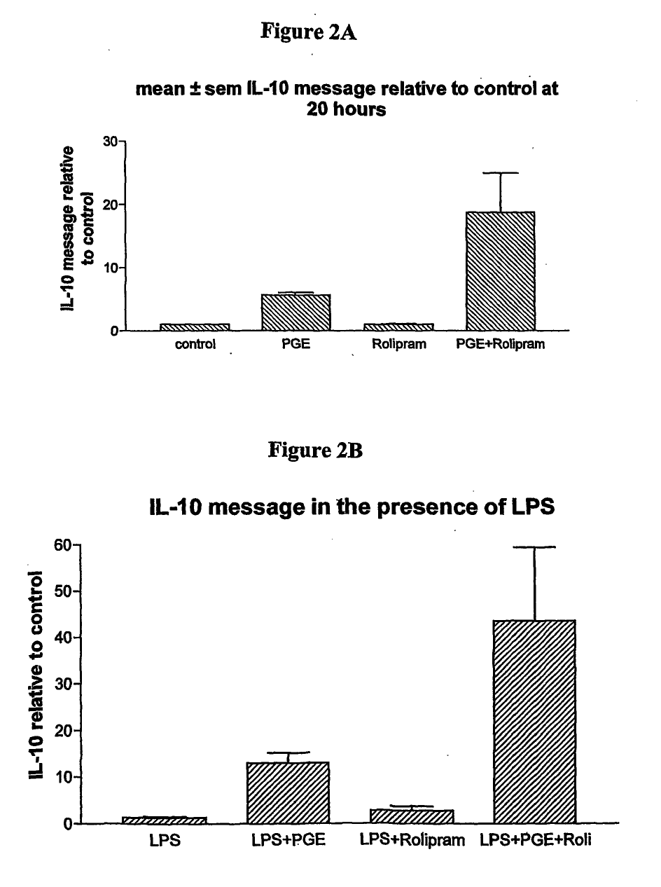

FIG. 2

FIG. 2A is a graph showing the effect of PGE and Rolipram on the production of IL-10 mRNA in U937 cells. FIG. 2B is a graph showing the effect of LPS, PGE and Rolipram on the production of IL-10 mRNA in U937 cells. FIG. 2C is a graph showing the effect of LPS, PGE and Rolipram on IL-10 release from U937 cells. FIG. 2D is a graph showing the effect of PGE and Rolipram on IL-10 release from U937 cells.

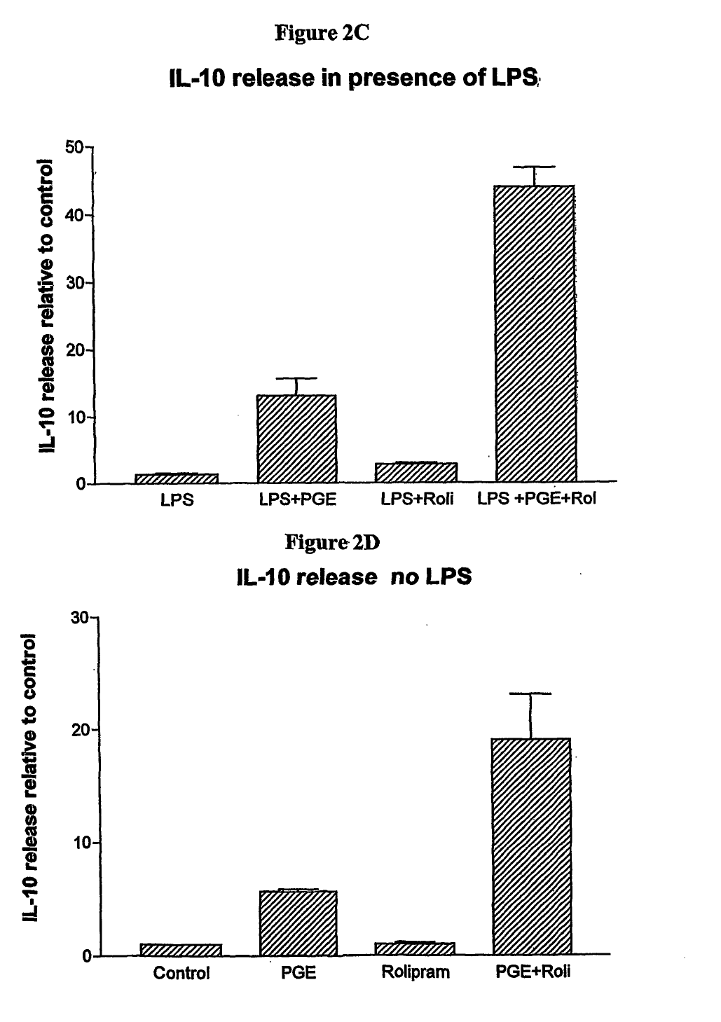

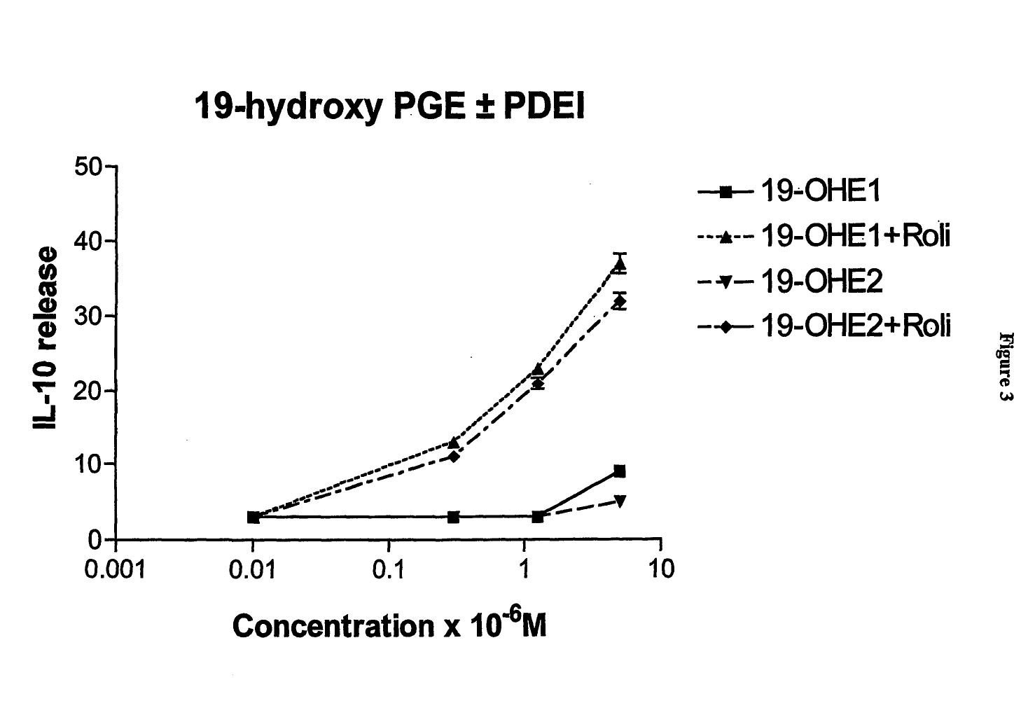

FIG. 3

A graph showing the effect of 19 hydroxy PGE1 and 19 hydroxy PGE2 on the stimulation of IL-10 in the presence and absence of rolipram.

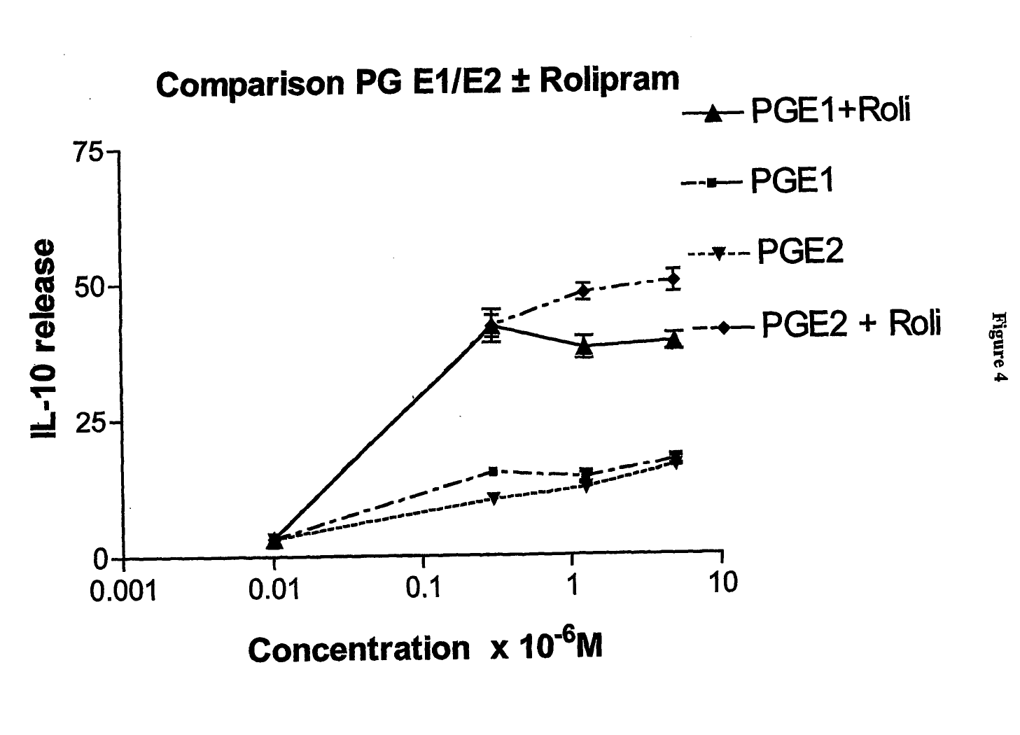

FIG. 4

A graph showing the effect of PGE1 and PGE2 on the stimulation of IL-10 in the presence and absence of rolipram.

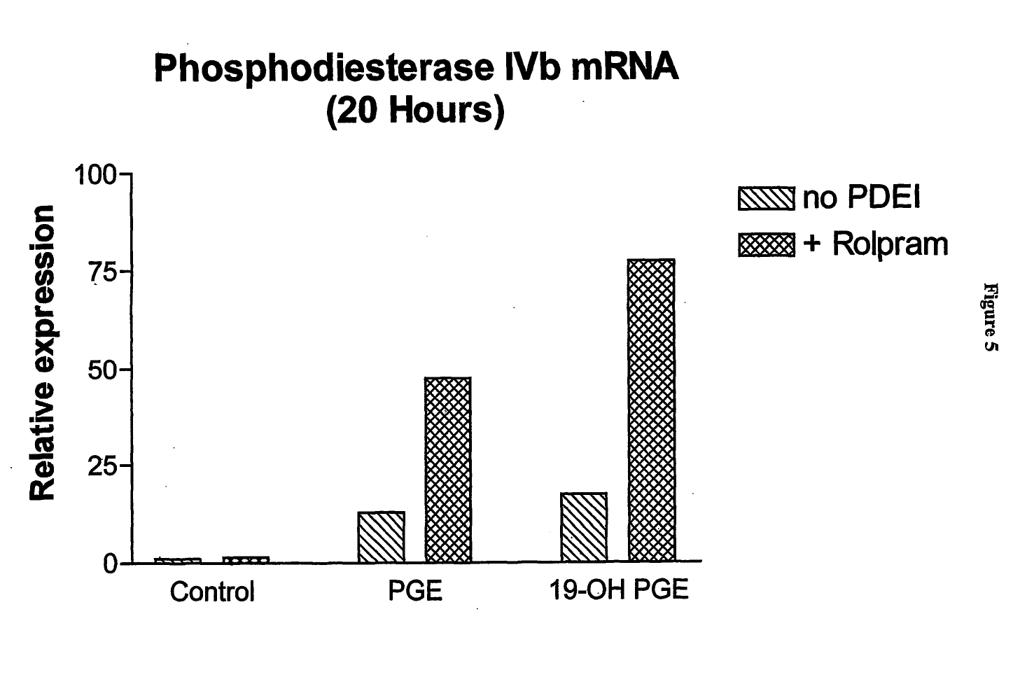

FIG. 5

A graph showing the effect of PGE and 19 hydroxy PGE on the production of phosphodiesterase IV b mRNA in the presence and absence of rolipram.

### EXAMPLE 1

### Effect of the Combination of PGE and Rolipram on IL-10 and IL-12 Production by U-937 (Promonocyte) Cells

U 937 (human monocyte cell line) cells were grown in RPMI (PAA Laboratories) medium with 10% fetal calf serum added (PAA Laboratories). Cells were treated with prostaglandin E2 at 10⁻6 Molar or with Interferon-γ at 10 ng/ml for 24 hours. Rolipram at 1 μg/ml and indomethacin at 10 μM was present in all wells. Cells were pelleted and the mRNA was extracted with Tri reagent (Sigma, Poole, UK). Total RNA was obtained by addition of chloroform and subsequent isopropanol precipitation. RNA was reverse transcribed with reverse transcriptase (Applied Biosystems) and random hexamers (Applied Biosystems). Probes and primers for IL-10 and IL-12 (p35) were designed using Primer Express (Applied Biosystems) and were as follows:

IL-12 p35 primers
CCACTCCAGACCCAGGAATG
TGTCTGGCCTTCTGGAGCAT
IL-12Probe
TCCCATGCCTTCACCACTCCCAA
IL-10, primers
CTACGGCGCTGTCATCGAT
TGGAGCTTATTAAAGGCATTCTTCA
IL-10 probe
CTTCCCTGTGAAAACAAGAGCAAGGCC

Template was amplified in a Taqman 7700 machine for 40 cycles using FAM/TAMRA dyes on the probe. The Applied Biosystems Kit was used to amplify and detect ribosomal (18S)RNA as a control. After 40 cycles the Ct (related to cycle number at which signal appears) for the FAM and the 18S (VIC) were recorded and absolute relative quantitation was achieved using the formula 2^{-ΔΔ}^{^{Ct}}.

The results of this experiment are described in the legend to FIG. 1. They show that there is a synergistic between a prostaglandin (PGE2) and a PDE inhibitor (rolipram) on the release of IL-10 from cells of the immune system and that there is a marked stimulation of IL-10 and inhibition of IL-12 in cells of the immune system when a prostaglandin (PGE2) and a PDE inhibitor (rolipram) are used in combination.

### EXAMPLE 2

### Stimulation of IL-10 Production is Achieved With or Without LPS

U 937 cells were grown in RPMI (PAA Laboratories) medium with 10% fetal calf serum added (PAA Laboratories). 2×10⁶ cells per flask were treated with prostaglandin E₂ at 10⁻6 Molar or with Rolipram (4×10⁻⁶) for 24 hours. Medium was removed at 20 hours and analysed by ELISA. A capture antibody (Pharmingen) was coated onto 96 well plates and culture medium was added each well. A standard curve was created with recombinant IL-10 protein. After incubation and washing, a biotin labelled monoclonal antibody (Pharmingen) was added and following incubation and washing, peroxidase labelled streptavidin was added. After washing a tetramethyl benzidine substrate was added and colour developed in proportion to IL-10 in the original sample/standard. Colour was read using a plate photometer (Labsystems, Multiskan). Mean concentrations (N=3) in controls with no lipopolysaccharide (LPS) were 38.2 pg/ml and in the presence of LPS (100 nM) they were 43.9 prostaglandin/ml.

After the incubation (20 hours), cells were pelleted and the mRNA was extracted with Tri-reagent (Sigma, Poole, UK). Total RNA was obtained by addition of chloroform and subsequent isopropanol precipitation. RNA was reverse transcribed with reverse transcriptase (Applied Biosystems) and random hexamers (Applied Biosystems). Probes and primers for IL-10 and IL-12 (p35) were designed using Primer Express (Applied Biosystems) and were as follows:

IL-12 p35 primers
CCACTCCAGACCCAGGAATG
TGTCTGGCCTTCTGGAGCAT
IL-12 probe
TCCCATGCCTTCACCACTCCCAA
IL-10 primers
CTACGGCGCTGTCATCGAT
TGGAGCTTATTAAAGGCATTCTTCA
IL-10 probe
CTTCCCTGTGAAAACAAGAGCAAGGCC

Template was amplified in a Taqman 7700 machine for 40 cycles using FAM/TAMRA dyes on the probe. The Applied Biosystems kit was used to amplify and detect ribosomal (18S)RNA (using VIC/TAMRA dyes) as an internal control in the same reaction tube. After 40 cycles the Ct (related to cycle number at which signal appears) for the FAM and the 18S (VIC) were recorded and absolute relative quantitation was achieved using the formula 2^{-ΔΔct} where Δ refers to the difference between the FAM and VIC signal related to an standard comparator included in each run.

### EXAMPLE 3

The effect of PGE1, PGE2, 19 hydroxy PGE1 and 19 hydroxy PGE2 on the stimulation of IL-10 in the presence and absence of rolipram was investigated as described above in Example 2. IL-10 levels were measured using an ELISA assay (R&D Ltd, Oxford). Measurements were taken in accordance with the manufacturer's instructions. Results are shown in FIGS. 3 and 4.

### EXAMPLE 4

The mRNA for phosphodiesterase IV-b was measured as described in Example 2 above mRNA was extracted after four hours of incubation. The concentration of the PGE was 1×10⁻⁶ and that of the 19-hydroxy PGE₂ was 5×10⁻⁶. The following primers and Taqman probe were used for quantitation of PDE IV b mRNA.

Forward
CCTTCAGTAGCACCGGAATCA
Reverse
CAAACAAACACACAGGCATGTAGTT
Probe
AGCCTGCAGCCGCTCCAGCC

Results are shown in FIG. 5. An increase in PDE activity follows both PGE and 19-hydroxy PGE application, which appears to be a direct negative feedback to reduce the effect of the stimulus. Use of a PGE and a type IV selective PDE inhibitor increases PDE message levels even further, but then the synthesised phosphodiesterase is nullified by the presence of the inhibitor.

### EXAMPLE 5

### Treatment of Rheumatoid Arthritis

A patient with rheumatoid arthritis is administered 800 μg misoprostol and 25 mg rolipram orally, daily.

### EXAMPLE 6

### Treatment of Demyelinating Disease

A patient with demyelinating disease is administered 800 μg misoprostol and 25 mg rolipram orally, daily.

1. A method of inducing tolerance to an antigen in a patient, the method comprising administering to the patient a prostaglandin or agonist thereof and a type IV selective phosphodiesterase (PDE) inhibitor.

2. A method according to claim 1 wherein the prostaglandin or agonist thereof is any one of a prostaglandin E, such as prostaglandin E₂ or an analogue thereof, dinoprostone, gemeprost, misoprostol, alprostadil, limaprost, butaprost, 11-deoxy PGE1, AH23848, AH130205, 19-hydroxy PGE1 or 19-hydroxy PGE2.

3. A method according to claim 1 wherein the prostaglandin is a 19-hydrozy PGE.

4. A method according to claim 1 wherein the type IV selective PDE inhibitor is any one of rolipram (4-[3-cyclopentyloxy-4-methoxyphenyl]-2-pyrrolidinone), CP80 633, CP102 995, CP76 593, Ro-20-1724 (4-[3-butoxy-4-methoxybenzyl]-2-imidazolidinone), denbufylline (1,3-di-n-butyl-7-(2-oxopropyl)-xanthine), CDP 840, RP 73401 or RS 33793.

5. A method according to claim 1 wherein the prostaglandin or agonist thereof and the type IV selective PDE inhibitor are administered simultaneously.

6. A method according to claim 1 wherein the prostaglandin or agonist thereof and/or type IV selective PDE inhibitor is administered locally at a site where tolerance is required.

7. A method according to claim 1 wherein the prostaglandin or agonist thereof and/or type IV selective PDE inhibitor is administered systemically.

8. A method according to claim 7 wherein the prostaglandin or agonist thereof and/or type IV selective PDE inhibitor is administered orally.

9. A method according to claim 7 wherein the prostaglandin or agonist thereof and/or type IV selective PDE inhibitor is administered as a suppository or capsule.

10. A method according to claim 9 wherein the suppository or capsule has an enteric coating for release of the prostaglandin or agonist thereof and/or type IV selective PDE inhibitor in the bowel of the patient.

11. A method according to claim 10 for treating inflammatory bowel disease.

12. A method according to claim 1 for combating a disease or condition associated with transplant rejection.

13. A method according to claim 12 wherein the disease or condition associated with transplant rejection comprises graft versus host disease or host versus graft disease.

14. A method according to claim 12 wherein the prostaglandin or agonist thereof and/or type IV selective PDE inhibitor are administered prior to the transplant.

15. A method according to claim 1 for treating an autoimmune disease other than rheumatoid arthritis.

16. A method according to claim 15 wherein the autoimmune disease is selected from primary myxoedema, thyrotoxicosis, pernicious anaemia, autoimmune atrophic gastris, Addison's disease, insulin-dependent diabetes mellitus (IDDM), Goodpasture's syndrome, myasthenia gravis, sympathetic ophthalmia, multiple sclerosis (MS), autoimmune haemolytic anaemia, idiopathic leucopenia, ulcerative colitis, dermatomyositis, scleroderma, mixed connective tissue disease, irritable bowel syndrome, systemic lupus erythromatosus (SLE), Hashimoto's disease, thyroiditis, Behcet's disease, coeliac disease/dermatitis herpetiformis, and demyelinating disease.

17. A method according to claim 1 for treating an allergic disease or condition in the patient.

18. A method according to claim 17 wherein the allergic disease or condition is allergic asthma.

19. A method according to claim 18 wherein the prostaglandin is a 19-hydroxy PGE, and wherein the 19-hydroxy PGE, and optionally the type IV selective PDE inhibitor, are administered via an aerosol to the bronchial tree or lungs of the patient.

20. A method according to claim 1 wherein the tolerance to the antigen is to treat an aberrant or undesired immune response to the antigen in the patient.

21. A method according to claim 20 wherein the aberrant or undesired immune response involves a deficiency in IL-10 production and/or an increase in IL-12 production.

22-43. (Canceled).

44. A therapeutic system for inducing tolerance to an antigen in a patient, the system comprising a prostaglandin or agonist thereof and a type IV selective PDE inhibitor.

45. A therapeutic system according to claim 44 wherein the type IV selective PDE inhibitor is in a preparation for systemic administration.

46. A therapeutic system according to claim 45 wherein the type IV selective PDE inhibitor is in a preparation for oral delivery.

47. A therapeutic system according to claim 44 wherein the prostaglandin or agonist is in a preparation for systemic administration, such as oral administration.

48. A therapeutic system according to claim 44 wherein the prostaglandin or agonist thereof is any one of a prostaglandin E, such as prostaglandin E₂ or an analogue thereof, dinoprostone, gemeprost, misoprostol, alprostadil, lamaprost, butaprost, 11-deoxy PGE1, AH23848, AH13205, 19-hydroxy PGE1 or 19-hydroxy PGE2.

49. A therapeutic system according to claim 44 wherein the prostaglandin is a 19-hydroxy PGE.

50. A therapeutic system according to claim 44 wherein the type IV selective PDE is any one of rolipram, CP80 633, CP102 995, CP76 593, Ro-20-1724, denbufylline, CDP 840, RP 73401 or RS 33793.

51. A therapeutic system according to claim 44 for combating a disease or condition associated with transplant rejection.

52. A therapeutic system according to claim 51 wherein the disease or condition associated with transplant rejection comprises graft versus host disease or host versus graft disease.

53. A therapeutic system according to claim 51 wherein the prostaglandin or agonist thereof and/or PDE inhibitor are for administration prior to the transplant.

54. A therapeutic system according to claim 44 for treating an autoimmune disease other than rheumatoid arthritis.

55. A therapeutic system according to claim 54 wherein the autoimmune disease is selected from primary myxoedema, thyrotoxicosis, pernicious anaemia, autoimmune atrophic gastris, Addison's disease, IDDM, Goodpasture's syndrome, myasthenia gravis, sympathetic ophthalmia, MS, autoimmune haemolytic anaemia, idiopathic leucopenia, ulcerative colitis, dermatomyositis, scleroderma, mixed connective tissue disease, irritable bowel syndrome, SLE, Hashimoto's disease and thyroiditis, Behcet's disease, coeliac disease/dermatitis herpetiformis, and demyelinating disease.

56. A therapeutic system according to claim 44 for treating an allergic disease or condition in the patient.

57. A therapeutic system according to claim 56 wherein the allergic disease or condition is allergic asthma.

58. A therapeutic system according to claim 57 wherein the prostaglandin is a 19-hydroxy PGE, and wherein the 19-hydroxy PGE, and optionally the type IV selective PDE inhibitor, are for administration to the bronchial tree or lungs of the patient via an aerosol.

59. A therapeutic system according to claim 44, wherein the tolerance to the antigen is to treat an aberrant or undesired immune response to the antigen in the patient.

60. A therapeutic system according to claim 59 wherein the aberrant or undesired immune response involves a deficiency in IL-10 production and/or an increase in IL-12 production.

61. (Canceled).
